(19) United States
(12) Patent Application Publication
(10) Pub. No.: US 2026/0071355 A1
(43) Pub. Date: Mar. 12, 2026

(54) CONDUCTIVE HYDROGEL FIBERS AND THE FABRICATION METHODS THEREOF

(71) Applicant: City University of Hong Kong, Hong Kong (HK)

(72) Inventors: Jinlian HU, Hong Kong (HK); Zhuang WANG, Hong Kong (HK)

(21) Appl. No.: 19/326,772

(22) Filed: Sep. 12, 2025

Related U.S. Application Data
(60) Provisional application No. 63/694,187, filed on Sep. 12, 2024.

(51) Int. Cl. D01F 6/14; A61B 5/27; A61F 2/08; D01D 10/02; D01F 1/09; D01F 11/06; D02G 3/02; D02G 3/44

(52) U.S. Cl. CPC D01F 6/14; A61B 5/27; A61F 2/08; D01D 10/02; D01F 1/09; D01F 11/06; D02G 3/02; D02G 3/448; A61B 2562/125; A61F 2002/0894; A61F 2230/0069; A61F 2240/001; D10B 2321/06; D10B 2401/02; D10B 2401/063; D10B 2401/16; D10B 2509/00

(57) ABSTRACT

A conductive hydrogel fiber including a hydrogel network formed from a water-soluble hydrogel polymer and conductive fillers dispersed therein is provided. The hydrogel network includes a porous structure having nanofibrillar network walls, the surfaces of which are provided with interconnected crystallized nanofibrils formed by concentration and aggregation of the hydrogel polymer. The conductive hydrogel fiber exhibits high electrical conductivity, excellent tensile strength, large elongation at break, high toughness, and low swelling in water.

### CROSS-REFERENCE TO RELATED APPLICATIONS

The present application claims priority from U.S. Provisional Patent application No. 63/694,187 filed Sep. 12, 2024; the disclosure of which is incorporated herein by reference in its entirety.

### FIELD OF THE INVENTION

The present invention generally relates to the field of functional polymeric materials. More specifically the present invention relates to the conductive hydrogel fibers and methods of their preparation.

### BACKGROUND OF THE INVENTION

Hydrogels are aqueous polymer networks widely employed as tissue engineering scaffolds, drug delivery carriers, and biological research model platforms due to their high water content, favorable biocompatibility, and human tissue-like mechanical properties. Conductive hydrogels are formed by incorporating conductive fillers—either ionic or electronic—into the hydrogel network. This modification retains the inherent mechanical properties of hydrogels while imparting electrical conductivity, enabling applications in wearable electronics, stretchable optical fibers, adhesives, and soft robotics.

Among conductive hydrogels, one-dimensional conductive hydrogel fibers offer structural advantages over two-dimensional films and three-dimensional bulk hydrogels. Their small diameters, light weight, high flexibility, and ease of weaving allow them to be reconfigured into multidimensional structures. These features make them attractive for artificial nerve fibers, artificial muscles, flexible robots, and bioelectrodes. For long-term, safe, and stable use in such applications, hydrogel fibers must exhibit high electrical conductivity, excellent water stability, and mechanical robustness under cyclic loading. However, researches in one-dimensional conductive hydrogel fibers—and their integration of these combined performance requirements—remain relatively limited.

Enhancing hydrogel conductivity typically requires introducing conductive substances such as carbon-based materials, metallic nanomaterials, conductive polymers, or ionic conductors. However, adding conductive fillers often creates a trade-off between electrical performance and mechanical strength. To minimize such compromise, established approaches involve carefully controlling filler content and employing microscale assembly techniques—such as laser selective sintering or solvent-evaporation phase separation—to form interconnected conductive networks. While effective in two-dimensional films and three-dimensional hydrogels, these techniques are incompatible with current hydrogel fiber manufacturing processes, including wet spinning, draw spinning, microfluidic spinning, and capillary polymerization.

Fibers produced via these conventional methods typically contain uniformly mixed polymer and conductive filler without refined microstructural control, resulting in relatively low electrical conductivity (<10 S/cm) and reduced mechanical performance compared to structured composites. Moreover, these processes often yield fibers with only simple physical or chemical crosslinking. Physically crosslinked fibers tend to exhibit poor water stability and limited strength, while chemically crosslinked fibers suffer from low tensile properties and brittleness. Although tough hydrogels have been developed by combining various dissipative mechanisms and structural designs, such methods—e.g., directional freezing, compositing, or mechanical stretching—are generally incompatible with fiber production.

Hydrogel fibers incorporating dissipative mechanisms such as double networks, dual crosslinking, or self-assembly may demonstrate toughness under single loading, but they typically fail to retain performance under repeated loading and are prone to fatigue fracture. Therefore, achieving conductive hydrogel fibers that simultaneously combine high conductivity, strength, stretchability, toughness, fatigue resistance, and water stability remains a significant and unresolved challenge, and the present invention aims to address this need.

### SUMMARY OF THE INVENTION

It is an objective of the present invention to provide the materials and the methods to solve the aforementioned technical problems.

In accordance with a first aspect of the present invention, a conductive hydrogel fiber is provided. Specifically, the conductive hydrogel fiber includes a hydrogel network, having a hydrogel polymer and conductive fillers dispersed therein with the concentration of the conductive fillers ranges from 1% to 5%. The hydrogel network has a porous structure having nanofibrils network walls, and the surfaces of the nanofibrillar network walls are provided with interconnected crystallized nanofibrils formed by concentration and aggregation of the hydrogel polymer. It is worth noting that the conductive hydrogel fiber has an electrical conductivity of 800-1,000 S/cm, a diameter of 1.5-0.3 mm, a tensile strength of 5-10 MPa, an elongation at bread of at least 300%, a toughness of 10-15 MJ/m², and a swelling rate in water of 8% to 52%.

In accordance with one embodiment, the crystallized nanofibrils comprise crystalline domains having a crystallinity ranging from 30%-40% and an average size ranging from 4-6 nm.

In accordance with another embodiment, the hydrogel polymer is water soluble and includes polyvinyl alcohol, polyacrylamide, polyethylene glycol, poly(2-hydroxyethyl methacrylate), cellulose derivatives, or any combinations thereof.

In accordance with yet another embodiment, the conductive fillers include metallic nanomaterials, carbon-based conductive materials, conductive polymers, ionic conductors, or any combinations thereof.

In accordance with yet another embodiment, the metallic nanomaterials include silver nanowires, silver nanoparticles, gold nanowires, copper nanowires, or combinations thereof.

In accordance with yet another embodiment, the fiber maintains at least 90% of its initial conductivity after 3,000 bending cycles.

In accordance with yet another embodiment, the fiber maintains at least 90% of its initial tensile strength after 5,000 tensile loading cycles at 3 MPa initial stress.

In accordance with a second aspect of the present invention, a method for fabricating the aforementioned conductive hydrogel fiber is provided. Particularly, the method includes: mixing a hydrogel polymer and conductive fillers to form a conductive hydrogel precursor solution; inducing phase separation of the conductive hydrogel precursor solution to form a preliminary fiber having a porous structure with nanofibrillar network walls; subjecting the preliminary fiber to a salting-out treatment with a salt solution to concentrate and aggregate the hydrogel polymer of the surfaces of the nanofibrillar network walls, thereby forming crystallized nanofibrils with crystalline domains and obtaining a salted-out fiber; drying and annealing the salted-out fiber at a temperature of 90-100° C. for 30-90 minutes to adjust the crystallinity and average crystalline domain size; and obtaining the conductive hydrogel fiber.

In accordance with one embodiment, the phase separation is induced by a freeze-thaw process.

In accordance with another embodiment, the hydrogel polymer is water soluble and comprises polyvinyl alcohol, polyacrylamide, polyethylene glycol, poly(2-hydroxyethyl methacrylate), cellulose derivatives, or any combinations thereof.

In accordance with yet another embodiment, the conductive fillers comprise metallic nanomaterials, carbon-based conductive materials, conductive polymers, ionic conductors, or any combinations thereof.

In accordance with yet another embodiment, the metallic nanomaterials comprise silver nanowires, silver nanoparticles, gold nanowires, copper nanowires, or any combinations thereof.

In accordance with yet another embodiment, the crystalline domains have a crystallinity of 30-40% and an average size of 4-6 nm.

In accordance with yet another embodiment, the salting-out treatment is performed using a salt solution having a concentration of 20-50 wt % sodium citrate or other inorganic salt.

In accordance with a third aspect of the present invention, a bioelectronic sensing system is provided. Specifically, the bioelectronic sensing system includes the aforementioned conductive hydrogel fiber configured as a bioelectrode; and a signal processing unit in electrical communication with the conductive hydrogel fiber.

In accordance with a fourth aspect of the present invention, an artificial axon for transmitting electrical signals in a bioelectronic or neuroprosthetic device is provided. Specifically, the artificial axon includes a hydrogel yarn made from a plurality of the aforementioned conductive hydrogel fibers.

In accordance with a fifth aspect of the present invention, a load-bearing artificial tissue is provided. Particularly, the load-bearing artificial tissue includes at least one hydrogel yarn formed by twisting together a plurality of the aforementioned conductive hydrogel fibers, and the load-bearing artificial tissue may be an artificial tendon or an artificial muscle.

### BRIEF DESCRIPTION OF THE DRAWINGS

Embodiments of the invention are described in more details hereinafter with reference to the drawings, in which:

FIGS. 1A-1D depict an overview of the method of fabricating the conductive hydrogel fibers and their electrical and mechanical properties, in which: FIG. 1A is a schematic illustration of the structural advancement in the conductive hydrogel fibers during the fiber forming process consists of freeze-thaw, salting-out, and drying-annealing; FIG. 1B is a schematic illustration of the hierarchical structure of the conductive hydrogel fibers; FIG. 1C depicts the structure of the crystallized nanofibrils; FIG. 1D shows the properties comparison between the optimized conductive hydrogel fibers (FS-90) and the freeze-thawed hydrogel fibers (FT); and FIG. 1E and FIG. 1F depict the structure evolution and phase separation of conductive hydrogel fibers during the preparation process;

FIGS. 2A-2I depict the morphology and structure evolution after different treatments, in which: FIG. 2A shows a scanning electron microscope (SEM) image of the conductive hydrogel fibers obtained after the freeze-thaw (FT) process, with a magnified view of the highlighted region; FIG. 2B illustrates a SEM image of the conductive hydrogel fibers obtained after the freeze-thaw and salting-out (FS) process, with a magnified view of the highlighted region; FIG. 2C depicts a SEM image of the conductive hydrogel fibers obtained after the freeze-thaw, salting-out and annealing for 90 minutes (FS-90) process, with a magnified view of the highlighted region; FIG. 2D depicts the differential scanning calorimetry (DSC) curves of FT, FS, and FS-0, FS-10, FS-90 fibers under dry state; FIG. 2E depicts the measured crystallinities in the dry and swollen states of FT fibers, FS fibers, FS-0, FS-10, and FS-90 fibers; FIG. 2F depicts the water content of FT, FS, FS-0, FS-10, and FS-90 fibers; FIG. 2G shows the representative SAXS profiles of FS-0 fibers and FS-90 fibers; FIG. 2H demonstrates the representative WXRD profiles of FT, FS, and FS-0, FS-10, and FS-90 fibers; and FIG. 2I illustrates the average crystalline domain size D of FT fibers, FS fibers, FS-0, FS-10, FS-90 fibers;

FIGS. 3A-3K depict the relationship between the structure and properties, in which FIG. 3A shows the stress-strain curves of the FT, FS, FS-0, FS-10, and FS-90 fibers; FIG. 3B depicts the stress, strain, and toughness of the FT, FS, FS-0, FS-10, and FS-90 fibers; FIG. 3C illustrates the successive loading-unloading cycling tests of the FS-90 fibers at strain ratios of 100% (left), 150% (middle), and 200% (right); FIG. 3D displays the images of the evolution of crack propagation profile in the FS-90 fiber during the tensile test; FIG. 3E and FIG. 3F respectively shows the schematic illustration of fatigue crack propagation in conductive hydrogel fibers with low and high crystallinities under cyclic loads; FIG. 3G depicts the conductivity of the FT, FS, FS-0, FS-10, and FS-90 fibers; FIG. 3H illustrates the conductivity changes of FS-90 fiber after immersion in water at different times; FIG. 3I shows the resistance changes of FS-90 fibers under successive loading-unloading and bending cycling tests; FIG. 3J displays the diameter changes of the conductive hydrogel fibers after immersion in water for 24 h and corresponding swelling ratios with an initial diameters defaulted to 1 mm; and FIG. 3K shows the schematic illustration of the mechanism of the crystallinity, swelling ratios, and conductivity between fibers with high crystallinity (left) and low crystallinity (right);

FIGS. 4A-4I depict the tunable properties and generality of conductive hydrogel fibers, in which FIG. 4A depicts an image of the FT fibers with different diameters; FIG. 4B shows the stress-strain curves of the FS-90 fibers with different diameters; FIG. 4C illustrates the conductivity of the FS-90 fibers with different diameters; FIG. 4D demonstrates the stress-strain curves of the FS-90 fibers with different mass ratios of AgNWs; FIG. 4E shows the conductivity of the FS-90 fibers with varying ratios of mass of AgNWs; FIG. 4F displays the resistance changes of the FS-90 fibers with different mass ratios of AgNWs; and FIGS. 4G-4I show the performance comparison with other known hydrogel fibers, including the conductivity vs. stretchability (FIG. 4G), conductivity vs. strength (FIG. 4H), conductivity vs. water content (FIG. 4I); and

FIGS. 5A-5I depict the multidimensional hydrogel structures and their applications, in which FIG. 5A is a schematic illustration of a single hydrogel fiber as artificial nerves; FIG. 5B is an illustration of the myelinated axon's structure and a single hydrogel fiber is connected to the circuits to test the potential signal-transmitting ability as an artificial nerve; FIG. 5C displays the input and output signal curves transmitted by a single conductive hydrogel fiber; FIG. 5D is a schematic showing hydrogel yarns composed of different numbers of hydrogel fibers; FIG. 5E is an illustration of the human muscle fiber and the images of the hydrogel yarns prepared with varying numbers of hydrogel fibers; FIG. 5F depicts the images of the hydrogel yarns used as the artificial tendon and the corresponding electrical signal change; FIG. 5G is an illustration of the hydrogel fabrics made from conductive hydrogel fibers; FIG. 5H is an illustration showing the hydrogel fabrics used as the ECG electrodes; and FIG. 5I displays the ECG signals measured with hydrogel fabric electrodes in air and underwater.

### DETAILED DESCRIPTION

In the following description, materials, methods and/or applications of conductive hydrogel fiber and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

As used herein, the term “salting-out” refers to a phase separation process in which the addition of a salt to a polymer solution reduces the solubility of the polymer in water, causing the polymer chains to aggregate or precipitate. In some embodiments, for PVA-based hydrogel fibers, adding a salt (e.g., sodium sulfate) changes the water structure and decreases the availability of free water molecules to solvate the polymer chains. This reduced hydration forces the PVA chains to interact more strongly with each other rather than with water, leading to aggregation and crystallization of the polymer. In the present invention, the effect is particularly localized to the pore walls, producing nanofibrillar crystalline structures that strengthen the hydrogel fiber and enhance its mechanical and water stability.

In accordance with a first aspect of the present invention, a conductive hydrogel fiber is provided. The conductive hydrogel fiber includes a hydrogel network formed from a hydrogel polymer in which conductive fillers are dispersed. The concentration of the conductive fillers is maintained within a range from 1% to 5% by weight, thereby ensuring both high electrical conductivity and mechanical flexibility. The hydrogel network features a porous structure composed of nanofibrillar network walls. The surfaces of these nanofibrillar network walls are covered with interconnected crystallized nanofibrils, which are formed through concentration and aggregation of the hydrogel polymer during fiber fabrication. The resulting conductive hydrogel fiber exhibits an electrical conductivity between 800 and 1,000 S/cm, a diameter in the range of 1.5 mm to 0.3 mm, a tensile strength from 5 to 10 MPa, an elongation at break of at least 300%, a toughness between 10 and 15 MJ/m², and a swelling rate in water within the range of 8% to 52%.

In some embodiments, the crystallized nanofibrils within the nanofibrillar network walls comprise crystalline domains having a crystallinity in the range of 30% to 40% and an average crystalline domain size between 4 nm and 6 nm. The hydrogel polymer is preferably water-soluble, and suitable examples include polyvinyl alcohol, polyacrylamide, polyethylene glycol, poly(2-hydroxyethyl methacrylate), cellulose derivatives, or combinations thereof. The conductive fillers may include metallic nanomaterials, carbon-based conductive materials, conductive polymers, ionic conductors, or combinations thereof, depending on the desired electrical and mechanical performance. In some preferred embodiments, the metallic nanomaterials are selected from silver nanowires, silver nanoparticles, gold nanowires, copper nanowires, or combinations thereof.

The conductive hydrogel fiber according to the present invention exhibits excellent durability under repeated deformation. In some embodiments, the fiber is capable of maintaining at least 90% of its initial electrical conductivity even after 3,000 bending cycles. Similarly, the fiber maintains at least 90% of its initial tensile strength after 5,000 tensile loading cycles conducted at an initial stress of 3 MPa.

In accordance with a second aspect of the present invention, a method for fabricating the aforementioned conductive hydrogel fiber is provided. The method begins by mixing a hydrogel polymer and conductive fillers to prepare a conductive hydrogel precursor solution. The hydrogel polymer is preferably water-soluble and may include polyvinyl alcohol, polyacrylamide, polyethylene glycol, poly(2-hydroxyethyl methacrylate), cellulose derivatives, or any combinations thereof. The conductive fillers may be selected from metallic nanomaterials, carbon-based conductive materials, conductive polymers, ionic conductors, or any combinations thereof. In certain preferred embodiments, the metallic nanomaterials include silver nanowires, silver nanoparticles, gold nanowires, copper nanowires, or combinations thereof, with silver nanowires being particularly advantageous due to their high intrinsic conductivity and excellent dispersion stability in aqueous systems.

The conductive hydrogel precursor solution is then subjected to a phase separation process to form a preliminary fiber having a porous structure with nanofibrillar network walls. In a preferred embodiment, this phase separation is induced by a freeze-thaw process in which the precursor solution is frozen and thawed in controlled cycles to promote the formation of interconnected nanofibrillar walls. This step imparts a highly porous, interconnected network that will later support the formation of the conductive pathways and crystalline domains.

Following the phase separation step, the preliminary fiber is subjected to a salting-out treatment using a salt solution, which functions to concentrate and aggregate the hydrogel polymer on the surfaces of the nanofibrillar network walls. This aggregation process leads to the formation of crystallized nanofibrils having well-defined crystalline domains. The salt solution used for the salting-out treatment preferably has a concentration of 20-50 wt % sodium citrate, although other inorganic salts may also be employed. This treatment produces a salted-out fiber with enhanced structural integrity and improved alignment of polymer chains within the nanofibrillar network walls.

The salted-out fiber is then subjected to a drying and annealing step, carried out at a temperature in the range of 90° C. to 100° C. for a duration of 30 to 90 minutes. This step adjusts both the overall crystallinity and the average crystalline domain size of the crystallized nanofibrils. In certain embodiments, the resulting crystalline domains exhibit a crystallinity in the range of 30% to 40% and an average size between 4 nm and 6 nm, which contributes to the fiber's high tensile strength, toughness, and resistance to swelling in aqueous environments.

Upon completion of the drying and annealing step, the finished conductive hydrogel fiber is obtained. The fiber retains a unique porous structure with nanofibrillar network walls whose surfaces are coated with interconnected crystallized nanofibrils, enabling an electrical conductivity of 800-1000 S/cm, a tensile strength of 5-10 MPa, an elongation at break of at least 300%, a toughness of 10-15 MJ/m², and excellent resistance to mechanical fatigue and swelling. This method provides a controllable and reproducible pathway for manufacturing high-performance conductive hydrogel fibers suitable for use in soft electronics, bioelectronics, wearable devices, and structural sensing applications.

In accordance with a third aspect of the present invention, a bioelectronic sensing system is provided. The bioelectronic sensing system incorporates the aforementioned conductive hydrogel fiber. In this configuration, the conductive hydrogel fiber is employed as a bioelectrode for acquiring electrical signals from a biological subject.

The bioelectrode is electrically connected to a signal processing unit, which is in direct electrical communication with the conductive hydrogel fiber. The signal processing unit is configured to receive, amplify, filter, and process electrical signals detected by the hydrogel fiber bioelectrode. This configuration enables high-fidelity acquisition and transmission of electrophysiological signals, such as electrocardiogram (ECG), electromyogram (EMG), or electroencephalogram (EEG) signals, both in dry and hydrated environments. The high conductivity of the hydrogel fiber ensures minimal signal loss, while its softness, flexibility, and biocompatibility allow for conformal contact with irregular tissue surfaces, reducing motion artifacts and improving signal quality. The integration of the conductive hydrogel fiber with the signal processing unit thus provides a bioelectronic sensing system suitable for wearable healthcare monitoring, neuroprosthetic interfaces, and other biomedical signal acquisition applications.

In accordance with a fourth aspect of the present invention, an artificial axon for transmitting electrical signals in a bioelectronic or neuroprosthetic device is provided. The artificial axon includes a hydrogel yarn that is formed by twisting or otherwise combining a plurality of the aforementioned conductive hydrogel fibers.

When multiple such conductive hydrogel fibers are combined into a hydrogel yarn, the resulting structure exhibits both high electrical conductivity and exceptional flexibility, allowing for efficient and stable transmission of electrical signals over extended periods and through complex, dynamic environments. The hydrogel yarn is capable of mimicking the structural and functional characteristics of natural axons, enabling it to serve as a soft, conductive pathway within bioelectronic or neuroprosthetic systems. Its high mechanical compliance and water content allow for close integration with biological tissue, minimizing immune responses and mechanical mismatch. The artificial axon can thus be used to transmit bioelectrical signals between neural tissue and electronic components, facilitating applications in nerve signal regeneration, brain-machine interfaces, and sensory or motor prosthetic devices, while maintaining signal fidelity and structural stability under repeated bending, stretching, and immersion in aqueous environments.

In accordance with a fifth aspect of the present invention, a load-bearing artificial tissue is provided. The load-bearing artificial tissue includes at least one hydrogel yarn formed by twisting together a plurality of the aforementioned conductive hydrogel fibers.

The hydrogel yarn that combines the flexibility, high water content, and biocompatibility of hydrogels with outstanding tensile performance and electrical conductivity. This yarn structure mimics the hierarchical arrangement of collagen bundles in natural load-bearing tissues, providing both strength and compliance under repetitive mechanical loading. The load-bearing artificial tissue of the present invention can be configured as an artificial tendon, capable of transferring mechanical forces between muscle and bone in prosthetic or robotic systems, or as an artificial muscle, capable of contracting or extending in response to mechanical or electrical stimuli. The conductive properties of the yarn also enable sensing and signal conduction functions, allowing the artificial tendon or muscle to serve not only as a mechanical actuator but also as a feedback component in biomechatronic systems.

### EXAMPLES

### Example 1. Preparation of Conductive Hydrogel Fibers

In this example, silver nanowires (AgNWs) are employed as conductive fillers and polyvinyl alcohol (PVA) chains as the hydrogel network to fabricate conductive hydrogel fibers. Briefly, AgNWs with a diameter of 80-90 nm and a length >80 μm are utilized. AgNWs are selected for their high intrinsic electrical conductivity (6.3×10⁵ S/cm) and good water dispersibility. To concentrate the AgNWs, a sedimentation method is employed in place of the commonly used centrifugation approach, as centrifugation—even at 1,000 rpm—can induce premature settling, leading to poor dispersion and clogging during injection.

PVA solution is prepared by dissolving PVA powder in deionized (DI) water, followed by heating to 95° C. under continuous stirring for 5 h. The mixture is then cooled to room temperature to yield a clear solution. In parallel, a 1.5 M sodium citrate solution is prepared by dissolving anhydrous sodium citrate powder in DI water and stirring magnetically for 1 h until clear.

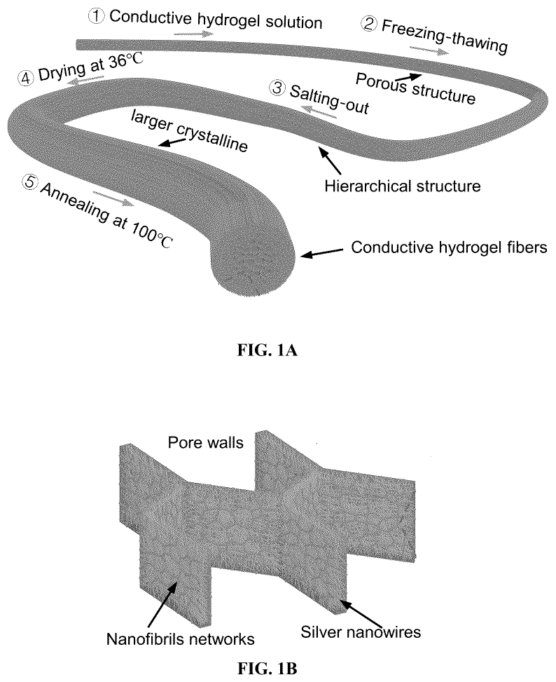

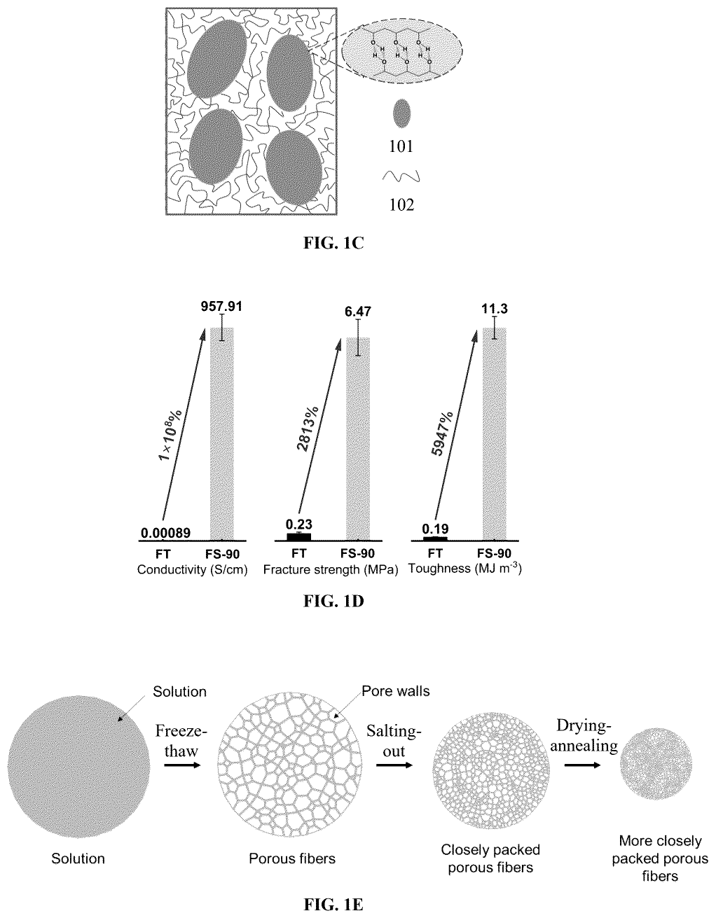

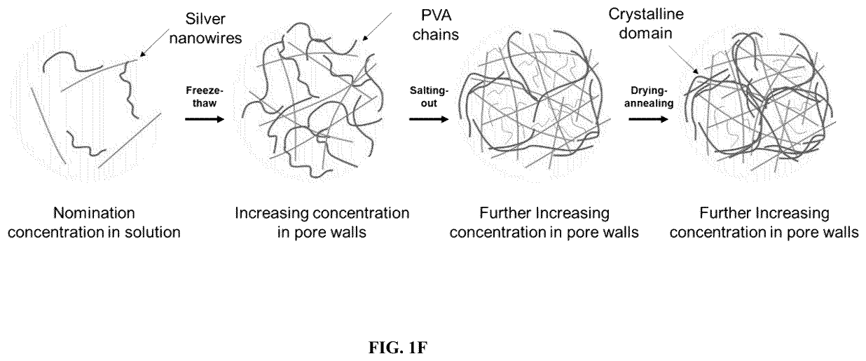

Initially, porous structures are formed by freezing and thawing the hydrogel precursor in a polytetrafluoroethylene (PTFE) tube, yielding freeze-thaw fibers (FT fibers). These are subsequently immersed in a salt solution to induce salting-out, producing hierarchical structures (FS fibers). A drying-annealing step at 100° C. for varying durations (0-90 min) is then applied to enhance crystallinity and enlarge crystalline domain size, yielding FS-0, FS-10, and FS-90 fibers. The preparation process is illustrated in FIG. 1A, while FIG. 1E and FIG. 1F depict the structural evolution and phase separation during fabrication. Specifically, the concentration of silver nanowires includes two aspects: the increase in volume density resulting from the reduction in fiber diameter, and the rise in the relative concentration of silver nanowires within the pore walls due to the crystallization of PVA. As shown in FIG. 1E, the volumetric concentration of silver nanowires increases progressively as the hydrogel fibers become more compact and their diameter decreases. In FIG. 1F, it can be seen that the relative enrichment of silver nanowires within the pore walls arises from the progressive phase separation of PVA chains. These PVA chains crystallize on the pore wall surfaces, forming nanofibrils that further densify the network and concentrate the silver nanowires in the pore walls.

During freeze-thawing, phase separation generates porous walls composed of AgNWs and PVA. Salting-out treatment concentrated and aggregated PVA chains, causing partial phase separation from pore walls and crystallization into interconnected nanofibrils at the surface (FIG. 1B). As shown in FIG. 1C, subsequent drying-annealing further concentrates and aggregates the PVA chains 101, increasing crystallinity and crystalline domain size of the crystalline domain 102 (FIG. 1C). The resulting hierarchical structure and crystalline domains contributed to mechanical energy dissipation, imparting high stretchability. The fibers also exhibit excellent conductivity, flexibility, and structural stability, maintaining conductivity under various deformations.

Compared to FT fibers, FS-90 fibers show marked improvements in conductivity, fracture strength, and toughness (FIG. 1D). This combined fabrication strategy is well-suited for large-scale production, and the prepared fibers demonstrate outstanding water stability.

In one embodiment, the PVA solution (10 wt %) is blended with AgNWs at the desired ratio (5 wt % AgNWs unless otherwise specified). The mixture is injected into polytetrafluoroethylene (PTFE) tubes of varying diameters and subjected to freeze-thaw cycling: freezing at −20° C. for 8 h, followed by thawing at 25° C. for 3 h, repeated three times. The resulting prefabricated FT fibers are removed from the tubes and subsequently treated by salting-out and drying-annealing. The annealed fibers are immersed in DI water to reach equilibrium swelling and stored in DI water until further use.

### Example 2. Morphography and Structure of the Conductive Hydrogel Fibers

The mechanical and electrical performance of the conductive hydrogel fibers is closely linked to their fine hierarchical structure and crystalline domains, both of which can be precisely tuned through the combined application of freeze-thaw, salting-out, and drying-annealing processes. To elucidate the contribution of each process, the morphology and structural evolution of the fibers are evaluated at different fabrication stages. The microstructure of the conductive hydrogel fibers is characterized using an FEI Quanta 250 e-SEM. Before the observation, the fibers are soaked in DI water for 24 h, and then the samples are freeze-dried using a LABFREEZ FD-10-MF freeze dryer. The freeze-dried samples are crisped with liquid nitrogen to expose their interiors.

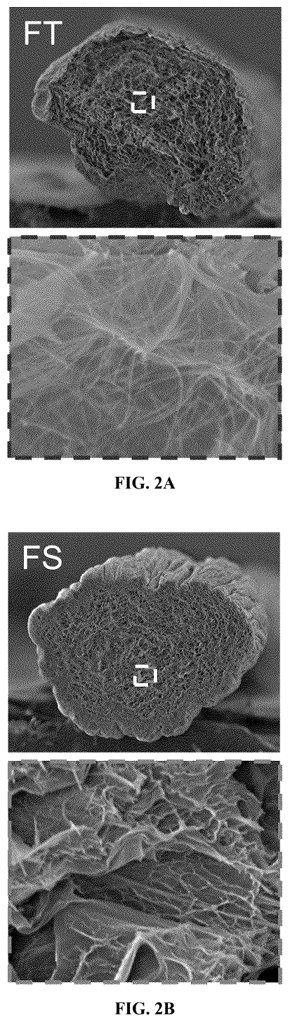

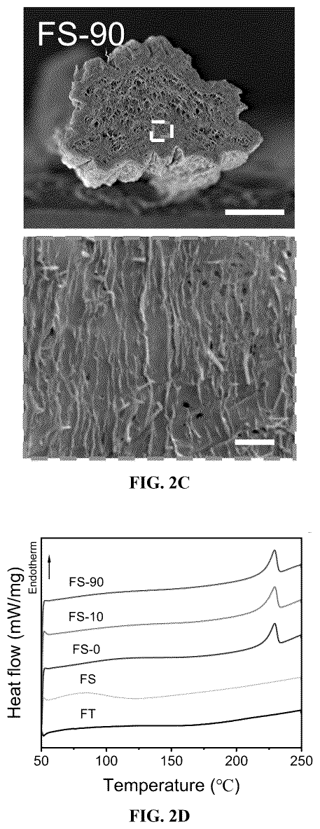

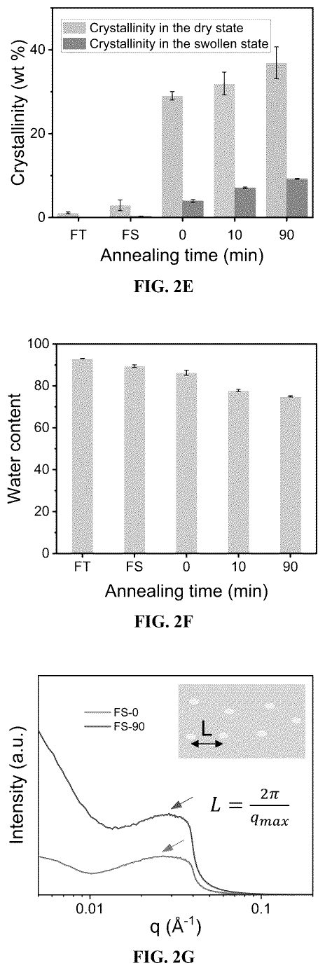

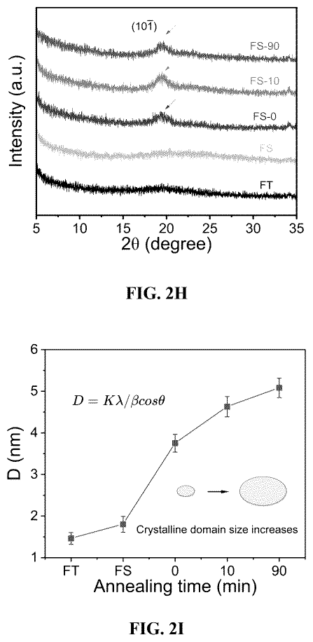

During the freeze-thaw process, PVA chains concentrate and aggregate to form pore walls, with AgNWs uniformly dispersed within the PVA matrix. The resulting FT fibers exhibit a loose, porous architecture with weak crosslinking (FIG. 2A). In contrast, FS fibers, obtained after salting-out in a high-concentration salt solution, have smaller diameters and denser packing (FIG. 2B). The salting-out effect induces further PVA phase separation from the pore walls, promoting crystallization into interconnected nanofibrils on the pore wall surfaces. Subsequent drying-annealing at 100° C. for 90 min produces FS-90 fibers with a more compact structure and further reduced diameter (FIG. 2C), as additional PVA chains aggregate and crystallize into nanofibrillar networks.

At the molecular scale, these combined processes markedly affect crystallinity and crystalline domain size. The crystallinity is measured based on the differential scanning calorimetry (DSC) measurements. First, to minimize the further formation of crystalline domains during the drying process, the amorphous chains in the conductive hydrogel fibers are fixed by excess chemical crosslinks induced by glutaraldehyde. The fixed conductive hydrogel fibers are soaked in DI water for 12 hours to remove the residual chemicals, and the samples are freeze-dried for testing. The weight of the dried samples is recorded (M). In a typical DSC measurement, the samples are subjected to a temperature increase from 50° C. to 250° C. at a rate of 20 K/min under a nitrogen atmosphere with a flow rate of 20 ml per minute. The enthalpy for the evaporation of any remaining water (H_{residual}) is determined by integrating the endothermic transition from 60° C. to 180° C. The heat flow curves display a narrow peak across the domains. Furthermore, the enthalpy for the melting of the crystalline domains per unit mass of the samples (H_{crystalline}) is determined by integrating the endothermic transition from 200° C. to 250° C. The masses of the residual water (M_{residual}) and the PVA crystalline (M_{crystaline}) can be calculated as follows:

$${M}_{residual} = M \times \frac{{H}_{residual}}{{H}_{water}^{0}} , \qquad ( 1 )$$

where, H_{water}⁰ is the latent heat of water evaporation, which is 2260 J/g.

$${M}_{crystalline} = M \times \frac{{H}_{crystalline}}{{H}_{crystalline}^{0}} , \qquad ( 2 )$$

where, H_{crystaline}⁰ is the enthalpy of fusion of 100 wt. % crystalline PVA, which is 183.6 J/g. Additionally, the starting solution has the same masses of AgNWs and PVA. As a result, the mass of PVA (M_{PVA}) is determined as follows:

$${M}_{PVA} = \frac{M - {M}_{residual}}{2} , \qquad ( 3 )$$

hence, the crystallinity (X) of the dry samples can be calculated as:

$$X = \frac{{M}_{crystalline}}{{M}_{PVA}} = \frac{2 {M}_{crystalline}}{M - {M}_{residual}} , \qquad ( 4 )$$

DSC shows only weak endothermic peaks between 200-250° C. for FT and FS fibers (FIG. 2D)), corresponding to low crystallinities of 1.1% and 2.9%, respectively (FIG. 2E, Table 1), attributable to limited aggregation during freeze-thawing and salting-out, as well as partial dissolution of crystallites in water. By contrast, drying-annealing for 0, 10, and 90 min produces prominent melting peaks of PVA crystallites and a substantial crystallinity increase from 29% to 36.9%, confirming that drying-annealing strongly promotes PVA chain aggregation. This trend parallels the reduction in water content from 93% (FT fibers) to 75% (FS-90 fibers) (FIG. 2F), as crystallization consumes amorphous PVA chains.

TABLE 1
Properties of hydrogel fibers fabricated with different parameters

| | Fabrication | Crystallinity | Crystalline | Stretchability | Strength | Toughness | Conductivity |
|---|---|---|---|---|---|---|---|
| Sample | methods | (%) | size (nm) | (%) | (MPa) | (MJ/m³) | (S/cm) |
| FT | Freeze- | 1.10 | 1.46 | 165.03 | 0.23 | 0.19 | 0.00089 |
| | thawing | | | | | | |
| FS | Freeze- | 2.93 | 1.80 | 226.28 | 1.08 | 1.30 | 66.89 |
| | thawing + | | | | | | |
| | salting-out | | | | | | |
| FS-0 | Freeze- | 29.04 | 3.75 | 252.92 | 1.40 | 1.82 | 181.95 |
| | thawing + | | | | | | |
| | salting-out + | | | | | | |
| | annealing | | | | | | |
| | at 36° C. | | | | | | |
| FS-10 | Freeze- | 31.95 | 4.63 | 292.23 | 3.44 | 5.57 | 701.50 |
| | thawing + | | | | | | |
| | salting-out + | | | | | | |
| | annealing | | | | | | |
| | at 100° C. | | | | | | |
| | for 10 min | | | | | | |
| FS-90 | Freeze- | 36.91 | 5.08 | 315.20 | 6.47 | 11.30 | 957.91 |
| | thawing + | | | | | | |
| | salting-out + | | | | | | |
| | annealing | | | | | | |
| | at 100° C. | | | | | | |
| | for 90 min | | | | | | |

Small-angle X-ray scattering (SAXS) reveals that the inter-crystallite distance decreases after 90 min drying-annealing, consistent with higher crystallinity and larger crystalline domains (FIG. 2G). Wide-angle X-ray diffraction (WXRD) confirms a dominant diffraction peak at 20=19.7°, corresponding to the (101) plane of semi-crystalline PVA (FIG. 2H). Scherrer analysis of the peak widths indicates that crystalline domain size increases from 1.5 nm (FT) to 5.2 nm (FS-90) (FIG. 2I).

All three processes—freeze-thaw, salting-out, and drying-annealing—promote crystallization and crystalline domain growth, with drying-annealing exerting the most pronounced effect.

### Example 3. Mechanical and Electrical Properties of the Conductive Hydrogel Fibers

Mechanical properties are critical for ensuring the long-term stability and safety of conductive hydrogel fibers in practical applications. For instance, the tensile properties of the hydrogel fiber are tested on a universal testing machine (Instron 5942 Micro Tester) at a rate of 20 mm/min.

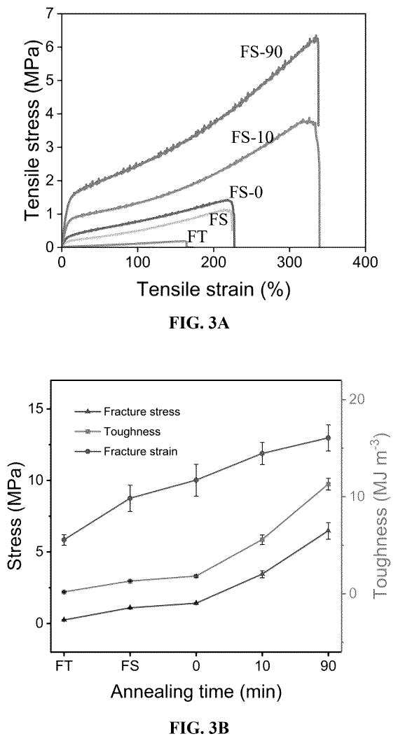

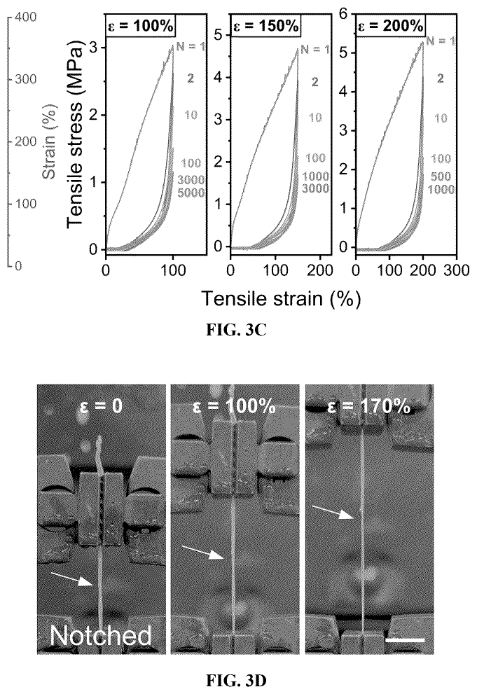

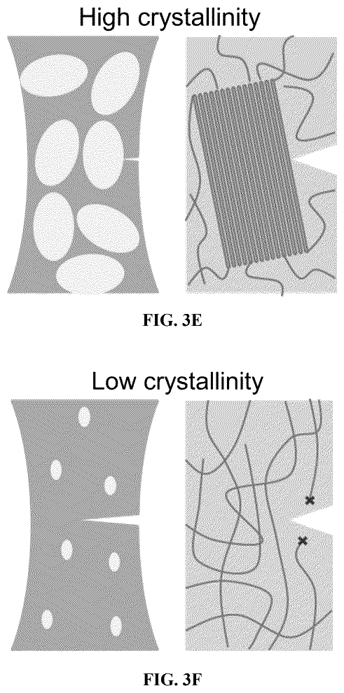

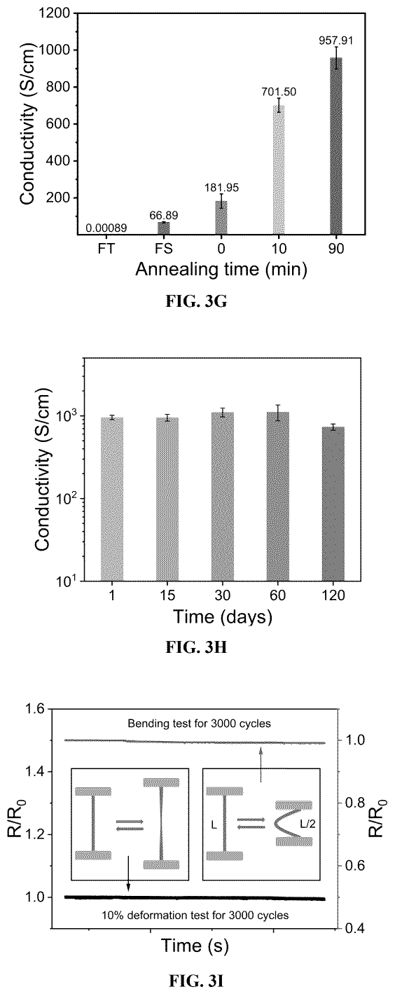

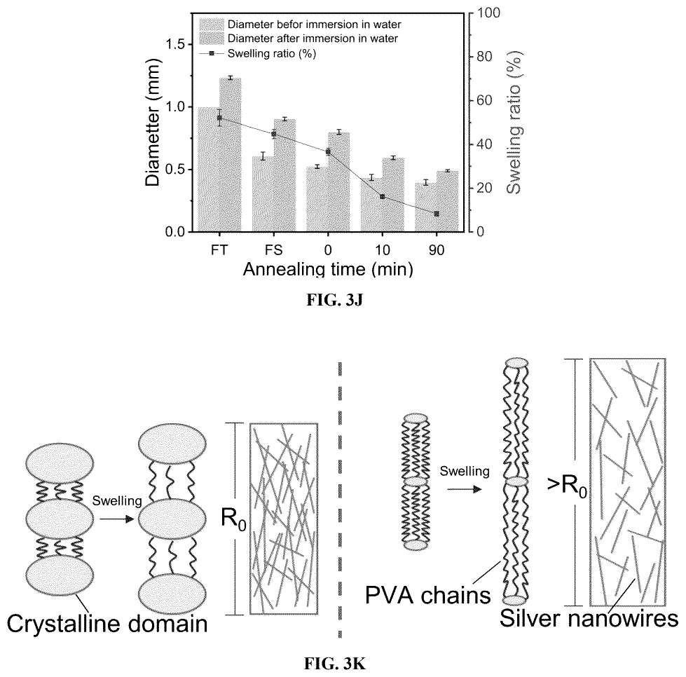

As shown in FIG. 3A, increasing crystallinity and crystalline domain size significantly enhances fiber strength and stretchability. Due to their low crystallinity, FT fibers exhibit the lowest tensile strength (˜0.15 MPa) and elongation (˜160%) (FIG. 3B and Table 1). In contrast, FS fibers show higher tensile stress and strain, attributed to their hierarchical structure with interconnected nanofibrils on the pore wall surfaces, which improve mechanical performance through energy dissipation and crack pinning. FS-90 fibers, with the highest crystallinity and largest crystalline domains, achieve a tensile strength of ˜6.2 MPa, elongation exceeding 300%, and a toughness of 12 MJ/m² compared to 0.2 MJ/m² for FT fibers. These enhancements arise from the synergistic effects of freeze-thaw, salting-out, and drying-annealing, which produce a densely packed hierarchical structure with abundant crystalline domains that facilitate efficient energy dissipation.

To ensure reliable long-term use in devices and equipment, conductive hydrogel fibers must retain their mechanical performance under repeated loading. The structural stability and fatigue resistance of FS-90 fibers are evaluated under cyclic loading with varying initial stresses. The fibers maintain stable performance over 5,000 cycles at 3 MPa (FIG. 3C), and even under a high initial stress of 5.5 MPa, they retained 2 MPa after thousands of cycles. Crack resistance is further assessed by introducing a notch (20% of fiber diameter) and subjecting the fibers to uniaxial tension. FS-90 fibers effectively suppressed crack propagation (FIG. 3D), whereas FT fibers experience rapid crack growth leading to premature fracture. This superior fatigue and fracture resistance stems from the high crystallinity and large crystalline domains of FS-90 fibers. Fatigue crack propagation in such fibers requires disruption of numerous, larger crystalline domains (FIG. 3E), which demands far more energy than breaking amorphous molecular chains in low-crystallinity fibers (FIG. 3F). Thus, increasing crystallinity and crystalline domain size markedly enhances fatigue resistance.

Electrical conductivity is critical for stable signal acquisition and transmission in conductive hydrogel fiber applications. The electrical conductivity is calculated by the equation:

$$σ = \frac{L}{R \times A} , \qquad ( 5 )$$

where σ is the conductivity of the conductive hydrogel fibers, L is the length, R is the resistance (measured by Keithley 2100), and A is the cross-section area of the conductive hydrogel fibers. The resistance change under strain is measured by elongating the sample with a universal testing machine (Instron 5942 Micro Tester) at a rate of 20 mm/min, using a gauge length of 10 mm. The resistance at both ends is logged using a Keithley 2100 digital multimeter. The resistance changes of hydrogel fiber under successive loading-unloading cycling tests and bending cycling tests are measured by successive stretching or bending the sample by using the universal testing machine (Instron 5942 Micro Tester) at a rate of 40 mm/min for loading tests and 200 mm/min for bending tests.

Control hydrogel fibers without AgNWs exhibit very low conductivity, confirming that conductivity arises from the interconnected network of silver nanowires. In the present invention, the freeze-thaw, salting-out, and drying-annealing processes collectively promote PVA chain aggregation and densify the hydrogel structure, facilitating the formation of continuous conductive pathways. Freeze-thaw concentrates AgNWs within interconnected pore walls, raising conductivity to 0.89 S/cm (FIG. 3G). Subsequent salting-out and drying-annealing further enhance AgNW packing, increasing conductivity to 67 S/cm (FS fibers) and 958 S/cm (FS-90 fibers). Among these processes, drying-annealing yields the largest improvement due to its pronounced crystallization effect. Beyond high conductivity, FS-90 fibers also demonstrate exceptional electrical stability, maintaining performance after four months in deionized water (FIG. 3H) and withstanding 3,000 bending and stretching cycles without significant degradation (FIG. 3I), highlighting their long-term structural and electrical reliability.

PVA crystalline domains can partially dissolve upon water immersion, compromising structural stability and causing hydrogel swelling or decomposition. Increasing crystallinity and crystalline domain size effectively suppresses swelling and enhances stability. This trend is evident from fiber diameter measurements before and after immersion (FIG. 3J): fibers with higher crystallinity exhibit denser packing and smaller initial diameters. Upon soaking, all fibers swell, but FS-90 fibers—with the highest crystallinity and largest crystalline domains—show the smallest final diameter and lowest swelling rate (FIG. 3K). At the molecular level, crystallization consumes amorphous PVA chains, which are otherwise prone to absorb water and expand. Thus, fibers with higher crystallinity contain fewer amorphous chains, leading to reduced swelling and better retention of electrical performance. Conversely, low-crystallinity fibers contain more amorphous chains, swell significantly, and suffer structural instability. Moreover, swelling disrupts conductive filler networks, further degrading electrical conductivity.

Overall, during hydrogel formation, AgNWs become concentrated and aggregated within the interconnected pore walls. As aggregation intensifies, the hydrogel network compacts, increasing AgNW—AgNW contact and forming robust, interconnected conductive pathways, thereby markedly improving electrical conductivity. At the microscopic level, this aggregation simultaneously promotes higher crystallinity and larger crystalline domains, with drying-annealing exerting the most significant effect. Elevated crystallinity and crystalline domain size enhance mechanical strength and fatigue resistance by introducing efficient energy dissipation mechanisms and increasing the fracture energy required for crack propagation. Additionally, these structural features suppress dissolution and swelling in aqueous environments, ensuring long-term structural integrity and stable electrical performance of the conductive hydrogel fibers.

### Example 4. Tunability and Customizability of Conductivity Hydrogel Fibers

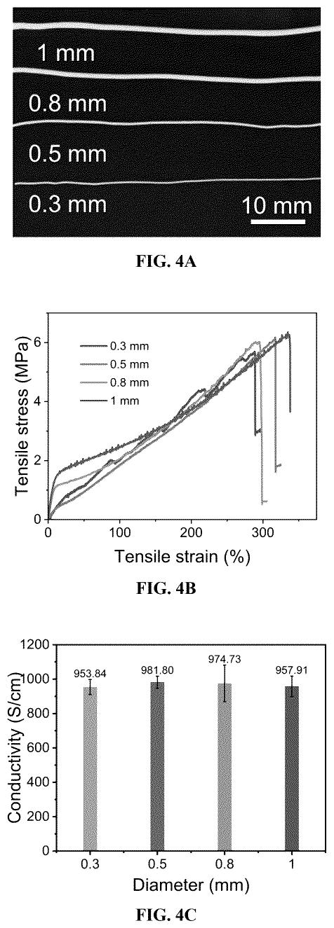

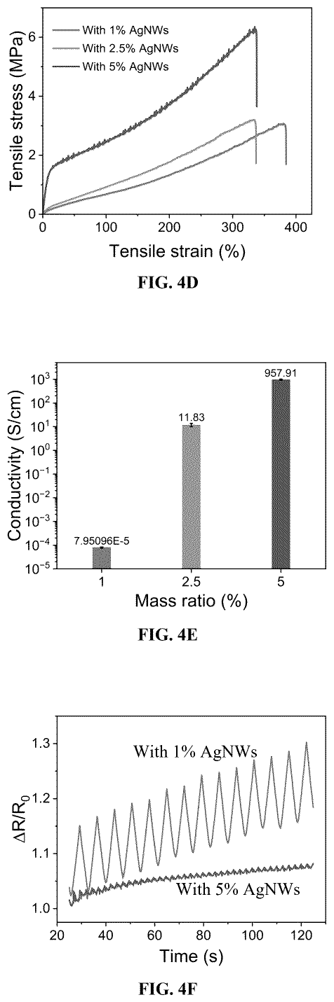

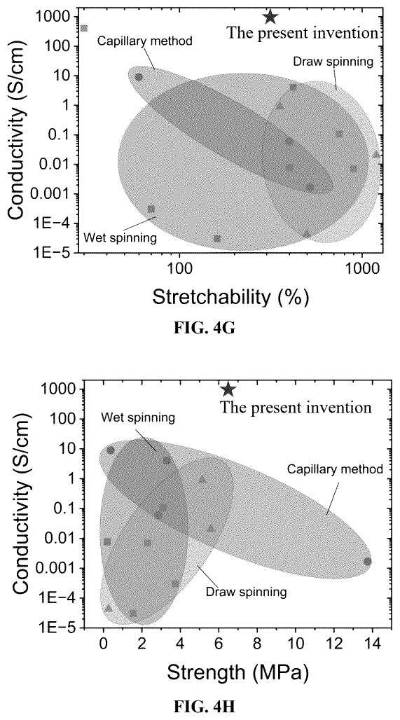

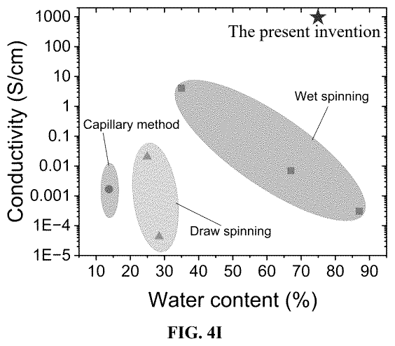

For different application scenarios, conductive hydrogel fibers with tunable properties—such as conductivity, modulus, and size—can better satisfy diverse requirements. However, most reported hydrogel fibers cannot achieve wide-range property modulation, as the spinning process is highly sensitive to the spinning solution. By contrast, the method of the present invention exhibits high adaptability, enabling facile tuning of the conductive hydrogel fibers' properties over an extensive range and allowing customization for specific applications. For example, freeze-thawed conductive hydrogel fibers with different diameters can be readily produced simply by employing PTFE tubes of varying sizes (FIG. 4A). The resulting fibers, despite their different diameters, show comparable tensile behavior (FIG. 4B) and conductivity (FIG. 4C), owing to the consistency of the materials, processing, and hierarchical structure. This demonstrates that the fabrication method is size-independent in building both hierarchical hydrogel structures and interconnected conductive networks, and can be generalized to other polymer or hydrogel fiber systems.

Furthermore, the electrical conductivity of the hydrogel fibers can be adjusted over a wide range by varying the AgNW content without altering the fiber preparation process. In composite systems, rigid fillers can enhance mechanical properties; accordingly, reducing the AgNW content results in decreased strength (FIG. 4D). At 1% AgNW content, the ultimate tensile stress decreases to approximately 3 MPa at approximately 400% strain, with the modulus dropping below 1 MPa. Electrical conductivity is also strongly dependent on AgNW content (FIG. 4E), decreasing from 958 S/cm (5% AgNWs) to 8×10⁻⁵ S/cm (1% AgNWs). This broad tunability enables the design of fibers for distinct functional roles: fibers with 5% AgNWs exhibit negligible resistance changes under 10% cyclic strain (FIG. 4F), making them suitable for bioelectrodes and electrical conductors, while fibers with 1% AgNWs show stable, strain-dependent resistance changes, enabling sensing applications. The simplicity and robustness of this strategy allow fiber size, conductivity, and mechanical properties to be tailored over a wide range, supporting large-scale production and diverse practical uses.

Compared with conductive hydrogel fibers produced by wet spinning, draw spinning, or template methods, the present strategy achieves a superior balance of conductivity, mechanical performance, and water stability. The optimized fibers combine high conductivity, excellent tensile strength and stretchability, water stability, and fatigue resistance. Notably, their electrical conductivity significantly exceeds that of previously reported conductive hydrogel fibers while maintaining desirable mechanical properties and water content (FIGS. 4G-4I). In addition, the process is environmentally friendly, requiring no organic solvents, coagulation baths, or toxic crosslinking agents.

### Example 5. Multidimensional Hydrogel Structure and their Applications

In addition to integrating ultrahigh conductivity, excellent mechanical performance, water stability, and fatigue resistance, the one-dimensional conductive hydrogel fibers of the present invention can be readily processed into multidimensional hydrogel structures, thereby expanding their applicability to diverse scenarios. Various prototypes have been fabricated to demonstrate their potential in related applications.

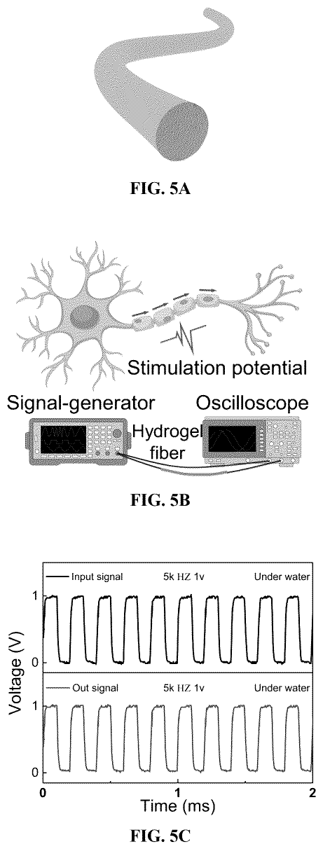

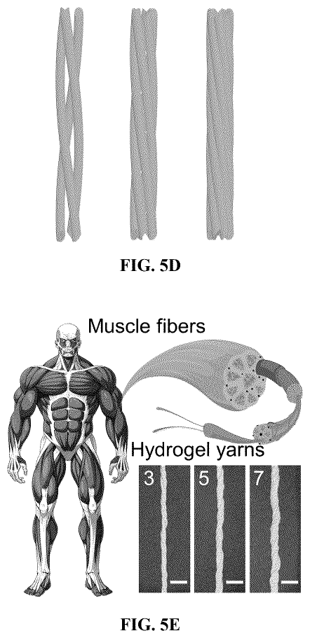

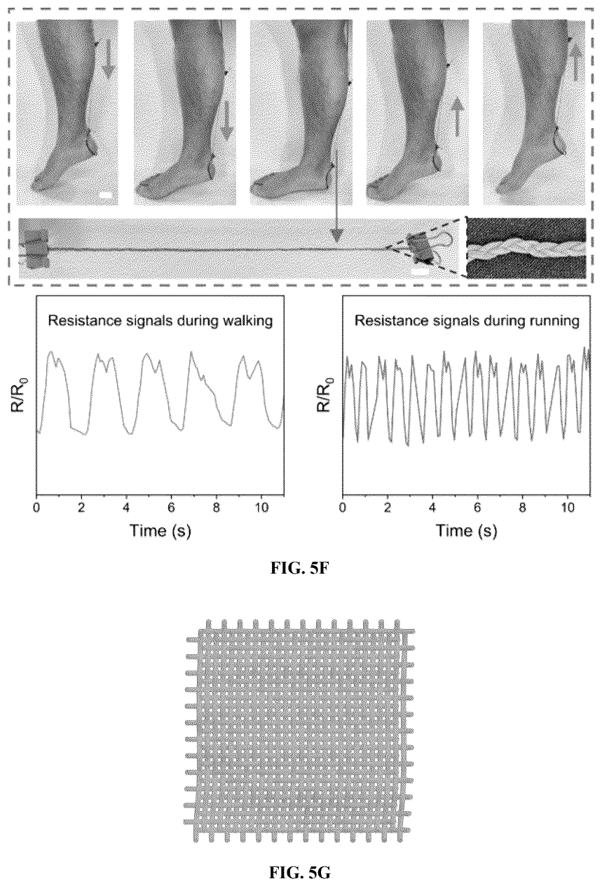

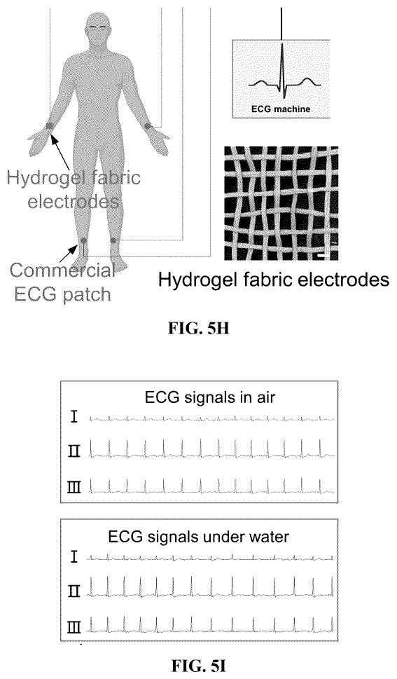

A single conductive hydrogel fiber, combining high conductivity with exceptional flexibility, serves as an ideal flexible conductor for use in underwater circuits, soft robotics, and artificial axons (FIG. 5A). As a proof-of-concept, the neural signaling process is simulated using a single conductive hydrogel fiber as an artificial axon (FIG. 5B). One end of the fiber is connected to an external signal generator as the input port, and the other end is connected to an oscilloscope as the output port. A time-dependent voltage signal is applied at the input, and the transmitted output is recorded. The signal waveforms before and after transmission show almost no phase shift or shape distortion, confirming excellent signal transmission capability (FIG. 5C).

Beyond single fibers, hydrogel yarns can be produced by twisting multiple conductive hydrogel fibers together (FIG. 5D and Figure S16, supporting information). These yarns mimic the hierarchical architecture of natural muscle fibers, offering tunable high mechanical strength and flexibility, making them well suited for load-bearing artificial tissues, artificial tendons, artificial muscles, hydrogel-based soft robotics, and bioelectronic applications (FIG. 5E). As a demonstration, a 20 cm hydrogel yarn braided from three plies of conductive fibers is integrated into a model leg as an artificial tendon. Owing to its excellent electrical stability and fatigue resistance, the yarn maintained sensing and signal conduction functions, highlighting its potential for underwater robotics and related devices (FIG. 5F).

Furthermore, the conductive hydrogel fibers can be woven into hydrogel fabrics, enabled by their outstanding flexibility and mechanical robustness (FIG. 5G). Compared with hydrogel films or blocks, hydrogel fabrics exhibit superior breathability, reducing skin discomfort during prolonged wear, and can conform well to irregular tissue surfaces, making them attractive as bioelectrode materials. As a demonstration, a hydrogel fabric is woven and used to record ECG signals (FIG. 5H). The results show stable, high-quality ECG recordings both in air and underwater (FIG. 5I).

Overall, these weavable, high-performance conductive hydrogel fibers—capable of being assembled into single fibers, yarns, or fabrics—offer a versatile platform for developing next-generation soft, conductive, and biocompatible devices across a wide spectrum of applications.

The present invention provides a simple yet effective strategy for fabricating conductive hydrogel fibers that combine ultrahigh conductivity, exceptional mechanical performance, excellent water stability, and high fatigue resistance. These synergistic properties arise from a sequential process of freeze-thaw, salting-out, and drying-annealing, which directs the controlled assembly of AgNWs and PVA chains. This process promotes the formation of interconnected conductive networks while significantly increasing the crystallinity and crystalline domain size. Furthermore, by adjusting process parameters, the size, conductivity, and mechanical properties of the hydrogel fibers can be readily tailored. Owing to their robust mechanical properties, the resulting fibers can be further processed into hydrogel yarns and woven fabrics for applications in soft robotics, bioelectronics, and wearable devices. This strategy not only enables scalable production of customized conductive hydrogel fibers but also broadens the design possibilities and practical applications of soft functional materials.

As used herein, terms “approximately”, “basically”, “substantially”, and “about” are used for describing and explaining a small variation. When being used in combination with an event or circumstance, the term may refer to a case in which the event or circumstance occurs precisely, and a case in which the event or circumstance occurs approximately. As used herein with respect to a given value or range, the term “about” generally means in the range of 10%, ±5%, ±1%, or ±0.5% of the given value or range. The range may be indicated herein as from one endpoint to another endpoint or between two endpoints. Unless otherwise specified, all the ranges disclosed in the present disclosure include endpoints. When reference is made to “substantially” the same numerical value or characteristic, the term may refer to a value within ±10%, ±5%, ±1%, or ±0.5% of the average of the values.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A conductive hydrogel fiber, comprising:
a hydrogel network, comprising a hydrogel polymer and conductive fillers dispersed therein, wherein the concentration of the conductive fillers ranges from 1% to 5%;
wherein the hydrogel network has a porous structure having nanofibrils network walls;
wherein surfaces of the nanofibrillar network walls are provided with interconnected crystallized nanofibrils formed by concentration and aggregation of the hydrogel polymer; and
wherein the conductive hydrogel fiber has an electrical conductivity of 800-1,000 S/cm, a diameter of 1.5-0.3 mm, a tensile strength of 5-10 MPa, an elongation at bread of at least 300%, a toughness of 10-15 MJ/m², and a swelling rate in water of 8% to 52%.

2. The conductive hydrogel fiber of claim 1, wherein the crystallized nanofibrils comprise crystalline domains having a crystallinity ranging from 30%-40% and an average size ranging from 4-6 nm.

3. The conductive hydrogel fiber of claim 1, wherein the hydrogel polymer is water soluble and comprises polyvinyl alcohol, polyacrylamide, polyethylene glycol, poly(2-hydroxyethyl methacrylate), cellulose derivatives, or any combinations thereof.

4. The conductive hydrogel fillers of claim 1, wherein the conductive fillers comprise metallic nanomaterials, carbon-based conductive materials, conductive polymers, ionic conductors, or any combinations thereof.

5. The conductive hydrogel fiber of claim 4, wherein the metallic nanomaterials comprise silver nanowires, silver nanoparticles, gold nanowires, copper nanowires, or any combinations thereof.

6. The conductive hydrogel fiber of claim 1, wherein the fiber maintains at least 90% of its initial conductivity after 3,000 bending cycles.

7. The conductive hydrogel fiber of claim 1, wherein the fiber maintains at least 90% of its initial tensile strength after 5,000 tensile loading cycles at 3 MPa initial stress.

8. A method for fabricating a conductive hydrogel fiber of claim 1, comprising:
mixing a hydrogel polymer and conductive fillers to form a conductive hydrogel precursor solution;
inducing phase separation of the conductive hydrogel precursor solution to form a preliminary fiber having a porous structure with nanofibrillar network walls;
subjecting the preliminary fiber to a salting-out treatment with a salt solution to concentrate and aggregate the hydrogel polymer of the surfaces of the nanofibrillar network walls, thereby forming crystallized nanofibrils with crystalline domains and obtaining a salted-out fiber;
drying and annealing the salted-out fiber at a temperature of 90-100° C. for 30-90 minutes to adjust the crystallinity and average crystalline domain size; and
obtaining the conductive hydrogel fiber.

9. The method of claim 8, wherein the phase separation is induced by a freeze-thaw process.

10. The method of claim 8, wherein the hydrogel polymer is water soluble and comprises polyvinyl alcohol, polyacrylamide, polyethylene glycol, poly(2-hydroxyethyl methacrylate), cellulose derivatives, or any combinations thereof.

11. The method of claim 8, wherein the conductive fillers comprise metallic nanomaterials, carbon-based conductive materials, conductive polymers, ionic conductors, or any combinations thereof.

12. The method of claim 11, wherein the metallic nanomaterials comprise silver nanowires, silver nanoparticles, gold nanowires, copper nanowires, or any combinations thereof.

13. The method of claim 8, wherein the crystalline domains have a crystallinity of 30-40% and an average size of 4-6 nm.

14. The method of claim 8, wherein the salting-out treatment is performed using a salt solution having a concentration of 20-50 wt % sodium citrate or other inorganic salt.

15. A bioelectronic sensing system comprising:
the conductive hydrogel fiber of claim 1, configured as a bioelectrode; and
a signal processing unit in electrical communication with the conductive hydrogel fiber.

16. An artificial axon for transmitting electrical signals in a bioelectronic or neuroprosthetic device, comprising a hydrogel yarn made from a plurality of the conductive hydrogel fibers of claim 1.

17. A load-bearing artificial tissue comprising at least one hydrogel yarn formed by twisting together a plurality of the conductive hydrogel fibers of claim 1, wherein the load-bearing artificial tissue comprises an artificial tendon and an artificial muscle.
